(19) United States
(12) Patent Application Publication
(10) Pub. No.: US 2005/0209656 A1
(43) Pub. Date: Sep. 22, 2005

(54) Method and apparatus for applying microcurrent to eyes

(76) Inventors: George Chiang Chuan Chen, Taipei (TW); Kuo Ching Chiang, Gueshan Township (TW)

Correspondence Address: DENNISON, SCHULTZ, DOUGHERTY;& MACDONALD, Suite 105, 1727 King Street, Alexandria, VA, 22314, US

(21) Appl. No.: 10/853,242

(22) Filed: May 26, 2004

(30) Foreign Application Priority Data
Mar. 19, 2004 (TW) 093107364

(51) Int. Cl. A61N 1/18

(52) U.S. Cl. 607/54

(57) ABSTRACT

A method for applying microcurrent to eyes, for use with a body, includes the steps of performing a current conduct process, then making a microcurrent loop between an eye and a corresponding hand to allow the microcurrent to apply to the eye according to the current conduct process of biochemical reaction. The present invention provides an apparatus for applying microcurrent to eyes, and the apparatus is used for the method.

### BACKGROUND OF THE INVENTION

1. Field of the Invention

This invention is generally related to a microcurrent biochemical application to eyes, especially to a method and apparatus for applying microcurrent to eyes, to make eyes generate biochemical reaction.

2. Description of Related Art

Recently, many researches verify microcurrent stimulation can improve the vascular function of retina. For example, ultraviolet rays shining in eyes produce freedom units to make eye pigment cells atrophy. Besides, if old men suffer hyperlipidemia, hypertension or diabetes, their retinas are apt to have difficulties in the circulation of blood, which causes deterioration diseases in maculae. That is, the patients' central eyesight having black shadow, destroying maculae to cause deterioration, making the central part of vision vague, so the eyesight declines gradually. Dr. Edward C. Kondrot, a Pittsburgh, Pa., ophthalmologist, in his work Miracle Eye Cure: “After patients have accepted microcurrent stimulation, the results in all test centers are positive. For example, 60% to 80% of the tested patients feel their visual abilities have improved. In addition, according to Dr. Paul's report, microcurrent stimulation can improve eyesight of 68% of the patients with dry macular degeneration. Microcurrent stimulation can also improve eyesight of 58% of the patients with wet macular degeneration.

Besides, according to researches, the reason of deterioration macular degeneration is the abnormality and declining of visual nerve units. That is, the electrical potential of visual cells decrease gradually, cell metabolism cycles are lower and cell replacing numbers decreases, so these make blood vessels lose elasticity and cause macular deterioration to fail eyesight. So, using microcurrent to stimulate maculae can reduce and cure deteriorating macular degeneration.

The medical proof that microcurrent can strengthen the eye function which is microcurrent can help the transmission of visual nerves, add electrical charge energy to visual nerve units directly, in order to make visual nerve cells active and accelerate. So, related muscles and blood vessels regain tenderness and elasticity gradually. Microcurrent stimulation follow Arndt-Schultz's rule: “Weak stimulation can promote bodies working normally, while strong stimulation may restrain or hinder action of body cells.”

Medical researches have verified currents can not only control pain effectively, but also promote the repair of tissue and strengthen cell function by stimulating the circulatory system. According to researches, current stimulation can promote the production and centralization of adenosine tri phosphate (ATP), help protein synthesis very greatly, supply enough energy for sodium pump and promote active transport mechanism, in order to make the metabolic waste in cells transport to blood and the nutrition in blood transport to cells, so stimulate cells to act and quicken metabolism. Fritz Lipmann, 1953 Nobel Prize winner, certified that ATP is the transporter and hoarder of all biochemical energy in cells. That is, the formation of ATP is by the burning of nutrition in biological cells. Afterward, ATP in biological bodies can cause cell material synthesis, muscular shrinks, transmission of nervous messages and many other physiological reactions, so ATP is called “cellular energy currency”. That is, if living things need energy, they have to use ATP. In 1980s, microcurrent has been widely applying to medicine in the U.S. According to researches, 68% of the patients under microcurrent stimulation have postponed and improved deteriorating macular degeneration.

Nevertheless, above researches only emphasize the biochemical effects of microcurrent, they do not provide the concrete method to lead microcurrent to eyes under safe and no-side-effect circumstances by using a specific amount of microcurrent to bring microcurrent effects into full play to strengthen the function of eyes.

### SUMMARY OF THE INVENTION

It is an object of the present invention to provide a method and apparatus for applying microcurrent to eyes. That is, microcurrent stimulates the specific acupuncture points and meridian of eyes and hands to generate ATP, and induces related biochemical mechanism, helping cells active and regenerate.

To achieve the above and other objects, the present invention provides a method for applying microcurrent to eyes, includes the steps of performing a current conduct process, and forming at least one microcurrent loop between at least one eye and at least one corresponding hand in a human body so as to cause the microcurrent to apply to the eye according to the current conduct process of biochemical reaction. After that, the tissue of the eye generates ATP.

In one embodiment of the present invention, the current conduct process includes the steps of sticking first medium to the eye on specific acupuncture points and specific meridian, and sticking second medium to the corresponding hand on specific acupuncture points and specific meridian, so that microcurrent flows into the eye and the corresponding hand through first medium and second medium.

To achieve the above and other objects, the present invention provides an apparatus for applying microcurrent to eyes, includes at least one signal wire, at least one medium unit and a host. Within, the medium unit couples with said signal wire to lead microcurrent into at least one eye and at least one corresponding hand in the human body. And, the host couples with the signal wire and outputs microcurrent into the medium unit with the signal wire, wherein microcurrent forms a microcurrent loop between the eye and the corresponding hand in the human body so as to cause microcurrent to apply to the eye.

In one embodiment of the present invention, first medium and second medium are electrode.

In one embodiment of the present invention, microcurrent outputs with a wave carrier.

In one embodiment of the present invention, the wave carrier frequency is between 5 KHz and 25 KHz.

In one embodiment of the present invention, microcurrent applies to the eye for a restricted time with the wave carrier including a kind of output frequency and a corresponding duty period, and applying alternatively for a corresponding duty time, and the microcurrent stops applying to the eye when the restricted time expires.

In one embodiment of the present invention, the range of output frequency is between 227 Hz and 333 Hz, the corresponding duty period is between 0.1 ms and 0.2 ms, and the corresponding duty time is between 0.1 minute and 2.8 minutes.

In one embodiment of the present invention, the range of output frequency is between 11 Hz and 35 Hz, the corresponding duty period is between 7 ms and 27 ms, and the corresponding duty time is between 1.5 minute and 4.1 minutes.

In one embodiment of the present invention, the range of output frequency is between 8 Hz and 27 Hz, the corresponding duty period is between 58 ms and 72 ms, and the corresponding duty time is between 9 minutes and 22 minutes.

In one embodiment of the present invention, the range of output frequency is between 0.01 Hz and 0.33 Hz, the corresponding duty period is between 1750 ms and 1950 ms, and the corresponding duty time is between 6 minutes and 22 minutes.

In one embodiment of the present invention, the restricted time is between 3 minutes and 33 minutes.

In one embodiment of the present invention, the microcurrent is between 10 μA (Micro-Ampere) and 2000 μA (Micro-Ampere).

In one embodiment of the present invention, the host includes a control unit and an output unit. The control unit outputs a control signal. The output unit for inputting the control signal and outputting the microcurrent couples with the control unit.

In one embodiment of the present invention, the host further includes an indicator unit coupled with the control unit. The indicator unit includes a power indicator for indicating power supply condition and a buzzer for sounding when the restricted time expires.

In one embodiment of the present invention, the host further includes an operating unit coupled with the control unit. The operating unit includes first intensity knob for controlling microcurrent outputting from first output receptacle and second intensity knob for controlling microcurrent outputting from second first output receptacle.

In one embodiment of the present invention, the medium unit includes first medium for sticking on the eye on specific acupuncture points and specific meridian, and second medium for sticking on the corresponding hand on specific acupuncture points and specific meridian, wherein microcurrent flows into the eye and the hand through first medium and first medium.

Conclusion the above, this invention provides the method and apparatus for applying microcurrent to eyes by using a microcurrent to stimulate eyeballs in order to produce adenosine tri phosphate and induce related biochemical mechanism, help cells metabolism and strengthen the function of eyes.

### BRIEF DESCRIPTION OF THE DRAWINGS

FIG. 1 is an electrical block diagram of the apparatus for applying microcurrent to eyes according to one embodiment of this invention; and

FIG. 2 illustrates one embodiment about a method for applying microcurrent to eyes on a human body.

### DETAILED DESCRIPTION OF THE PREFERRED EMBODIMENTS

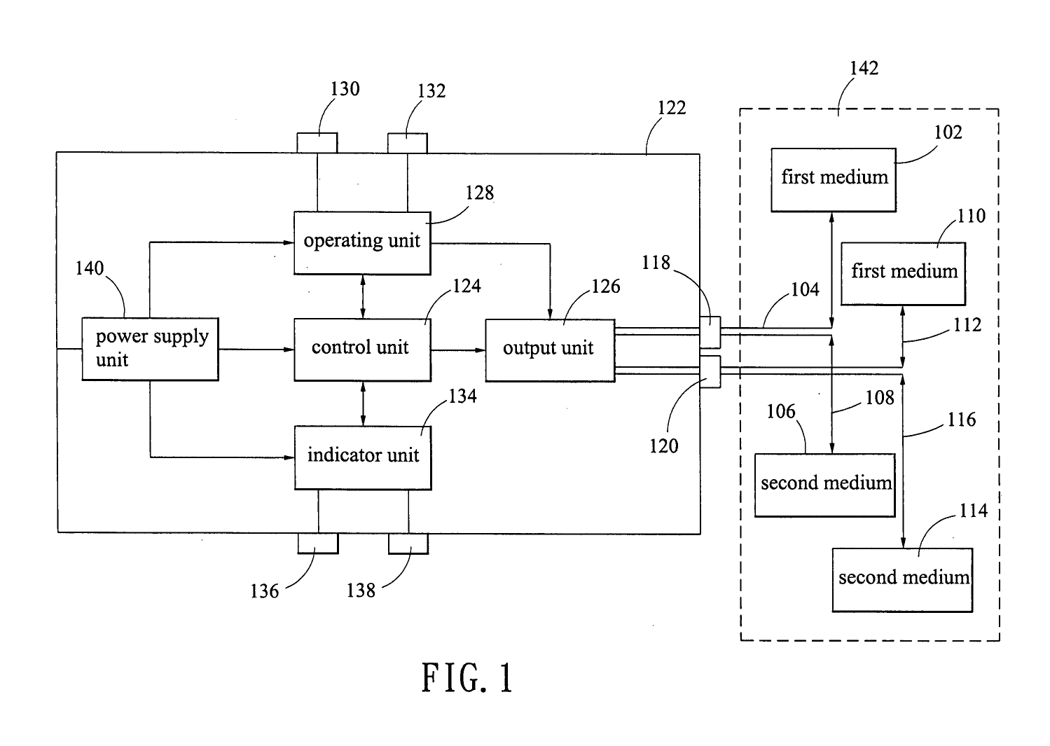

Please refer to FIG. 1, it is the electrical block diagram of the apparatus for applying microcurrent to eyes according to one embodiment of this invention. This apparatus includes a medium unit 142, signal wires 104, 108, 112, 116, and a host 122. The medium unit 142 includes first medium 102, second medium 106, first medium 110 and second medium 114. Further, host 122 includes first output receptacle 118, second output receptacle 120, a control unit 124, a output unit 126, a operating unit 128, first intensity knob 130, second intensity knob 132, a indicator unit 134, a buzzer 136, a power indicator 138 and a power supply unit 140. Within, power indicators 138 indicate the condition of power supply.

Within, first medium 102, second medium 106, first medium 110 and second medium 114 are electrodes. First medium 102 couples signal wire 104 electrically, and signal wire 104 couples first output receptacle 118 electrically. Further, second medium 106 couples signal wire 108 electrically, and signal wire 108 couples first output receptacle 118 electrically. Within, first medium 110 couples signal wire 112 electrically, and signal wire 112 couples second output receptacle 120 electrically. Further, second medium 106 couples signal wire 108 electrically, and signal wire 108 couples first output receptacle 118 electrically. Within, first medium 110 couples signal wire 112 electrically, and signal wire 112 couples second output receptacle 120 electrically. Further, second medium 114 couples signal wire 116 electrically, and signal wire 116 couples first output receptacle 120 electrically.

Further, first output receptacle 118 and second output receptacle 120 couple output receptacle 126 electrically. Besides, output unit 126 couples control unit 124 electrically. And, control unit 124 couples operating unit 128 and indicator 134 electrically. Within, operating unit 128 couples first intensity knob 130 and second intensity knob 132 electrically. In addition, indicator unit 134 couples buzzer 136 and power indicator 138 electrically. On the other side, power supply unit 140 couples control unit 124, operating unit 128 and indicator unit 134. Power supply unit 140 supplies direct current to host 122.

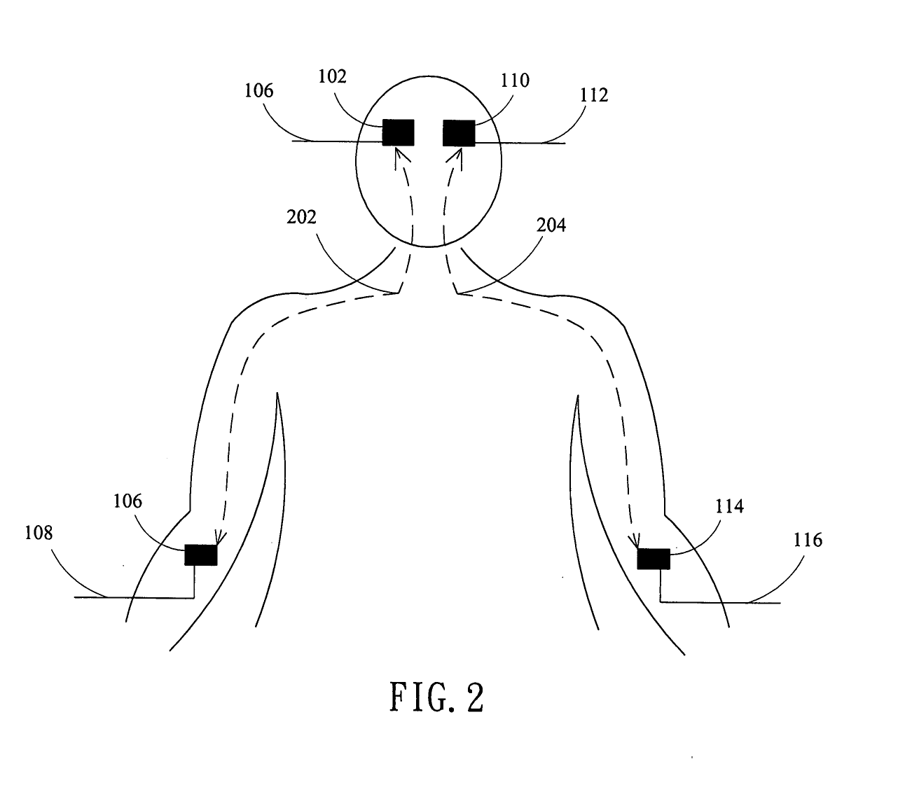

The following indicates the operation of this invention. Please refer to FIG. 2, it illustrates one embodiment about a method for applying microcurrent to eyes on a human body. This method includes the following steps: Before using this apparatus, a user performs one current conduct process. Then, by this current conduct process, microcurrent forms two microcurrent loops between the left eye and the corresponding left hand and between the right eye and the corresponding right hand in human body, which can cause microcurrent to apply to eyes.

The above current conduct process includes the following steps: For one user, the user sticks first medium 102 to right eye and sticks second medium 104 to the corresponding right hand. Further, the user sticks first medium 110 to left eye and sticks second medium 114 to the corresponding left hand. Then, turn on the power. Control unit 124 outputs a control signal to output unit 126 to make output unit 126 output first microcurrent to first output receptacle 118 and output unit 126 outputs second microcurrent to second output receptacle 118. Afterward, first output receptacle 118 transports first microcurrent through signal wire 104 to first medium 102. Then, there is an electric potential between first medium 102 and second medium 106, so microcurrent flows into a human body through first medium 102. After, microcurrent flows into second medium 106. Then, microcurrent flows into first output receptacle 118 through second medium 106. Finally, microcurrent flows back to output unit 126 through first output receptacle 118. Therefore, the above microcurrent can form first microcurrent loop 202.

In similar circumstances, after second output receptacle 120 transports second microcurrent to first medium 110 through signal wire 112, the microcurrent can form second microcurrent loop 204. Besides, microcurrent continually performs polarity change every time, which is between 0.3 minute to 3 minutes, to make current direction change. According to the above, in human bodies, first microcurrent loop 202 can stimulate the specific acupuncture points (e.i. a term of Chinese Doctor, vital points recognized in karate and acupuncture, certain parts in the human body where nerve centers are supposed to be located) and meridian of right eyes and right hands. Similarly, second microcurrent loop 204 can stimulate the specific acupuncture points and meridian of left eyes and left hands. Within, the specific acupuncture points of eyes are Tsuan-Chu (B2), Ching-Ming (B1), Tung-Tzu-Jiao (GB-1), Tayan, May-Chong, Cheng-Chi (S1), etc.; the specific one of hands is Ney-Gwan (T5).

The above control unit 124 is a build-in memory microprocessor, using general purpose input/output (GPIO) to output the above control signal. That is, the control signal includes a specific wave carrier. Further, control unit 124 outputs the specific wave carrier into output unit 126 to perform digital-to-analog converting and signal amplifying, etc. Then, output unit 126 outputs the specific wave carrier through first output receptacles 118 and 120.

From the above, the microcurrent applies to eyes for a restricted time with at least one wave carrier including various output frequencies and the corresponding duty period and applying alternatively for a corresponding duty time, and the microcurrent stops applying to said eye when said restricted time expires. In the instance, microcurrent outputs in four forms. The wave carrier frequency is between 5 kHz and 25 kHz. The first microcurrent form is, if the output frequency scope is between 227 Hz and 333 Hz, the corresponding duty period is between 0.1 ms and 0.2 ms, and the corresponding duty time is between 0.1 minute and 2.8 minutes. The second microcurrent form is, if the output frequency scope is between 11 Hz and 35 Hz, the corresponding duty period is between 7 ms and 22 ms, and the corresponding duty time is between 1.5 minute and 4.1 minutes. The third microcurrent form is, if the output frequency scope is between 8 Hz and 27 Hz, the corresponding duty period is between 58 ms and 72 ms, and the corresponding duty time is between 9 minutes and 22 minutes. The fourth microcurrent form is, if the output frequency scope is between 0.01 Hz and 0.33 Hz, the corresponding duty period is between 1750 ms and 1950 ms, and the corresponding duty time is between 6 minutes and 22 minutes.

The above four microcurrent forms can use one to four at the same time depending on circumstances, but must stop in restricted time between 3 minutes and 33 minutes. When the restricted time is up, the buzzer 136 sounds a period of alarm sound. Further, microcurrent is between 10 μA (Micro-Ampere) and 2000 μA (Micro-Ampere). On controlling microcurrent capacity, the first intensity knob 130 controls the microcurrent capacity of first output receptacle 118 and the second intensity knob 132 controls the microcurrent capacity of the second output receptacle 120. For example, first intensity knob 130 couples a variable resistance, adjusting the variable resistance can change the microcurrent flowing. It worth notice that the above values are the best results of the experiment to make eyes generates positive biochemical reaction. If microcurrent applying to eyes produces too little energy, it can't take expecting effect. If microcurrent applying to eyes generates too much energy, it may do harm to human body.

It is to be understood that the performance herein is just for example, the apparatus of the present invention mentioned above can use only first medium and second medium for one eye, or use some groups of first and second mediums to allow some other people use the apparatus at the same time. In addition, forms and types of microcurrent can also be changed as desired. So, the applying method of the present invention can be varied, changed, and modified by those skilled in the art depending on circumstances.

In conclusion, as mentioned above, this invention claims a method and apparatus for applying a various specific ranges of weak microcurrents to eyes to stimulate visual nerves to make cells within eyes to be more active and regenerated, so, this invention is safe and no-side-effect.

While the invention herein disclosed has been described by means of specific embodiments, numerous modifications and variations could be made thereto by those skilled in the art without departing from the scope and spirit of the invention set forth in the claims.

1. A method for applying microcurrent to eyes, for use with a human body, comprising:
performing a current conduct process; and
forming at least one microcurrent loop between at least one eye and at least one corresponding hand in said human body so as to cause said microcurrent to apply to said eye according to said current conduct process.

2. The method of claim 1, wherein said current conduct process comprises:
sticking at least one first medium to said eye on at least one specific acupuncture point and specific meridian; and
sticking at least one second medium to said corresponding hand on at least one specific acupuncture point and specific meridian;
wherein said microcurrent flows into said eye and said corresponding hand through said first medium and said second medium.

3. The method of claim 2, wherein said first medium is an electrode.

4. The method of claim 2, wherein said second medium is an electrode.

5. The method of claim 2, wherein said specific acupuncture point corresponding to said eye is Tsuan-Chu (B2).

6. The method of claim 2, wherein said specific acupuncture point corresponding to said eye is Ching-Ming (B1).

7. The method of claim 2, wherein said specific acupuncture point corresponding to said eye is Tung-Tzu-Jiao (GB-1).

8. The method of claim 2, wherein said specific acupuncture point corresponding to said eye is Tayan.

9. The method of claim 2, wherein said specific acupuncture point corresponding to said eye is May-Chong.

10. The method of claim 2, wherein said specific acupuncture point corresponding to said eye is Cheng-Chi (S1).

11. The method of claim 2, wherein said specific acupuncture point corresponding to said hand is Ney-Gwan (T5).

12. The method of claim 1, wherein said microcurrent outputs with a wave carrier.

13. The method of claim 12, wherein said wave carrier frequency is between 5 KHz and 25 KHz.

14. The method of claim 1, wherein said microcurrent applies to said eye for a restricted time with at least one wave carrier comprised of at least one output frequencies and at least one corresponding period and outputting alternatively for a corresponding time, and said microcurrent stops applying to said eye when said restricted time expires.

15. The method of claim 14, wherein said output frequency is between 227 Hz and 333 Hz, said corresponding period is between 0.1 ms and 0.2 ms, and the corresponding time is between 0.1 minute and 2.8 minutes.

16. The method of claim 14, wherein said output frequency scope is between 11 Hz and 35 Hz, said corresponding period is between 7 ms and 27 ms, and the corresponding time is between 1.5 minute and 4.1 minutes.

17. The method of claim 14, wherein said output frequency scope is between 8 Hz and 27 Hz, said corresponding period is between 58 ms and 72 ms, and the corresponding time is between 9 minutes and 22 minutes.

18. The method of claim 14, wherein said output frequency is between 0.01 Hz and 0.33 Hz, said corresponding period is between 1750 ms and 1950 ms, and the corresponding time is between 6 minutes and 22 minutes.

19. The method of claim 14, wherein said restricted time is between 3 minutes and 33 minutes.

20. The method of claim 14, wherein an alarm sound is further provided when said restricted time expires.

21. The method of claim 1, wherein said microcurrent is between 10 μA (Micro-Ampere) and 2000 μA (Micro-Ampere).

22. The method of claim 14, wherein said microcurrent changes polarity between 0.3 minute and 3 minutes every time.

23. A apparatus for applying microcurrent to eyes, for use with a human body, comprising:
at least one signal wire;
at least one medium unit coupled with said signal wire to lead said microcurrent into at least one eye and at least one corresponding hand in said human body; and
a host coupled with said signal wire and outputting said microcurrent into said medium unit with said signal wire, wherein said microcurrent forms at least one microcurrent loop between said eye and said corresponding hand in said human body so as to cause said microcurrent to apply to said eye.

24. The apparatus of claim 23, wherein said host comprises:
a control unit for outputting a control signal; and
an output unit coupled with said control unit, for inputting said control signal and outputting said microcurrent.

25. The apparatus of claim 23, wherein said host further comprises an indicator unit coupled with said control unit, said indicator unit comprise:
a power indicator for indicating power supply condition; and
a buzzer for sounding when said restricted time expires.

26. The apparatus of claim 23, wherein said host further comprises an operating unit coupled with said control unit, said operating unit comprises:
at least one first intensity knob for controlling said microcurrent outputting from at least one first output receptacle; and
at least one second intensity knob for controlling said microcurrent outputting from at least one second output receptacle.

27. The apparatus of claim 23, wherein said medium unit comprises:
at least one first medium for sticking on said eye on at least one specific acupuncture point and specific meridian; and
at least one second medium for sticking on said corresponding hand on at least one specific acupuncture point and specific meridian;
wherein said microcurrent flows into said eye and said hand through said first medium and said first medium.

28. The method of claim 27, wherein said first medium is an electrode.

29. The method of claim 27, wherein said second medium is an electrode.

30. The method of claim 27, wherein said specific acupuncture point corresponding to said eye is Tsuan-Chu (B2).

31. The method of claim 27, wherein said specific acupuncture point corresponding to said eye is Ching-Ming (S1).

32. The method of claim 27, wherein said specific acupuncture point corresponding to said eye is Tung-Tzu-Jiao (GB-1).

33. The method of claim 27, wherein said specific acupuncture point corresponding to said eye is Tayan.

34. The method of claim 27, wherein said specific acupuncture point corresponding to said eye is May-Chong.

35. The method of claim 27, wherein said specific acupuncture point corresponding to said eye is Cheng-Chi (S1).

36. The method of claim 27, wherein said specific acupuncture point corresponding to said hand is Ney-Gwan (T5).

37. The method of claim 23, wherein said microcurrent outputs with a wave carrier.

38. The method of claim 23, wherein said wave carrier frequency is between 5 KHz and 25 KHz.

39. The method of claim 23, wherein said microcurrent applies to said eye for a restricted time with at least one wave carrier comprised of at least one output frequencies and at least one corresponding period and outputting alternatively for a corresponding duty time, and said microcurrent stops applying to said eye when said restricted time expires.

40. The method of claim 39, wherein said output frequency is between 227 Hz and 333 Hz, said corresponding duty period is between 0.1 ms and 0.2 ms, and the corresponding duty time is between 0.1 minute and 2.8 minutes.

41. The method of claim 39, wherein said output frequency is between 11 Hz and 35 Hz, said corresponding duty period is between 7 ms and 27 ms, and the corresponding duty time is between 1.5 minute and 4.1 minutes.

42. The method of claim 39, wherein said output frequency is between 8 Hz and 27 Hz, said corresponding period is between 58 ms and 72 ms, and the corresponding time is between 9 minutes and 22 minutes.

43. The method of claim 39, wherein said output frequency is between 0.01 Hz and 0.33 Hz, said corresponding duty period is between 1750 ms and 1950 ms, and the corresponding duty time is between 6 minutes and 22 minutes.

44. The method of claim 39, wherein said restricted time is between 3 minutes and 33 minutes.

45. The method of claim 39, wherein an alarm sound is further provided when said restricted time expires.

46. The method of claim 23, wherein said microcurrent is between 10 μA (Micro-Ampere) and 2000 μA (Micro-Ampere).

47. The method of claim 23, wherein said microcurrent changes polarity between 0.3 minute and 3 minutes every time.
